(19) United States
(12) Patent Application Publication
(10) Pub. No.: US 2026/0334839 A1
(43) Pub. Date: Oct. 29, 2026

(54) STRATEGY FOR ACQUIRING BIOELECTRICAL SIGNALS WIRELESSLY

(71) Applicant: Michael Sampson, Oolitic, IN (US)

(72) Inventor: Michael Sampson, Oolitic, IN (US)

(21) Appl. No.: 19/648,416

(22) Filed: Apr. 15, 2026

Related U.S. Application Data
(60) Provisional application No. 63/793,323, filed on Apr. 23, 2025.

(51) Int. Cl. A61B 5/243; A61B 5/00; A61B 5/28; A61B 5/339; A61B 5/353; A61B 5/355

(52) U.S. Cl. CPC A61B 5/243; A61B 5/0006; A61B 5/28; A61B 5/339; A61B 5/353; A61B 5/355; A61B 5/746

(57) ABSTRACT

An electrocardiogram (ECG) system may be capable of generating, acquiring, and processing a signal that is influenced by the interaction between an induced or sensed magnetic and electromagnetic fields and the bioelectrical characteristics of a patient's tissue in contact with or adjacent to, an electrode. The resulting signal could include features that correlate with physiological activity and can be extracted through various signal processing methods for various purposes. Alternatively, the resulting signal could be displayed.

### TECHNICAL FIELD

The present disclosure relates generally to a strategy for wirelessly acquiring bioelectrical signals, and more particularly to a wireless electrocardiogram system and a related methodology for operating the same.

### BACKGROUND

Conventional bioelectrical signal acquisition systems, such as electrocardiogram monitoring systems, typically rely on electrodes connected via wired leads to external recording units. Electrocardiogram monitoring systems generally depend on some type of tethered configuration to maintain continuous signal acquisition, and could also require direct skin preparation, gel application, and electrode placement. The presence of the wired leads in some instances, absent mitigation, introduce several operational constraints, including, for example, limited mobility for the patient, increased risk of lead dislodgement, and entanglement during routine activities. For instance, in a clinical application, the use of wired systems could complicate patient handling, hinder mobility during rehabilitation, and increase the burden on healthcare personnel during setup and maintenance of signal acquisition.

Other limitations could include an impact on data quality and completeness, particularly, for instance, in ambulatory monitoring where motion and wire lead tension may result in intermittent signal dropout or motion-induced artifacts. Wired systems could also reduce patient adherence in long-term or at-home monitoring scenarios due to discomfort, visibility, or interference with daily activities. While current bioelectrical signal acquisition systems are used due to their established workflows, there is always the need for non-invasive bioelectrical technologies that can offer comparable signal fidelity without the use of wires.

### SUMMARY

In one aspect, operating a wireless electrocardiogram includes generating an energizing electromagnetic field to induce an electric current in an electrode attached to a patient. Operating a wireless electrocardiogram system also includes wirelessly detecting a change in an electrical property of the electrode attached to the patient. Operating a wireless electrocardiogram system further includes producing a signal indicative of electrocardiographic activity of the patient based on the detected change.

In another aspect, detecting bioelectrical activity includes placing at least one electrode in direct electrical contact with the body of a patient. Detecting bioelectrical activity also includes energizing an electromagnetic coil to wirelessly induce electric current in the at least one electrode. Detecting bioelectrical activity further includes monitoring a sensing electromagnetic field, and wirelessly detecting a change in bioelectrical activity of the body of the patient based on the monitoring the sensing electromagnetic field.

In yet another aspect, an electrocardiogram system includes an energizing coil and contact pads structured for attaching to a patient. The electrocardiogram system also includes a sensing coil structured to wirelessly monitor electrical activity of the contact pads induced by an electromagnetic field produced by the energizing coil.

### BRIEF DESCRIPTION OF DRAWINGS

FIG. 1 is a perspective view of a patient resting on a support structure and a cassette, according to one embodiment;

FIG. 2 is a top plan view of an electrode, according to one embodiment;

FIG. 3 is a sectioned view, in perspective, of the electrode shown in FIG. 2, according to one embodiment;

FIG. 4 is a sectioned view, in perspective, of a cassette, according to one embodiment; and

FIG. 5 is an exemplary depiction of a controller in communication with a target, according to one embodiment.

### DETAILED DESCRIPTION

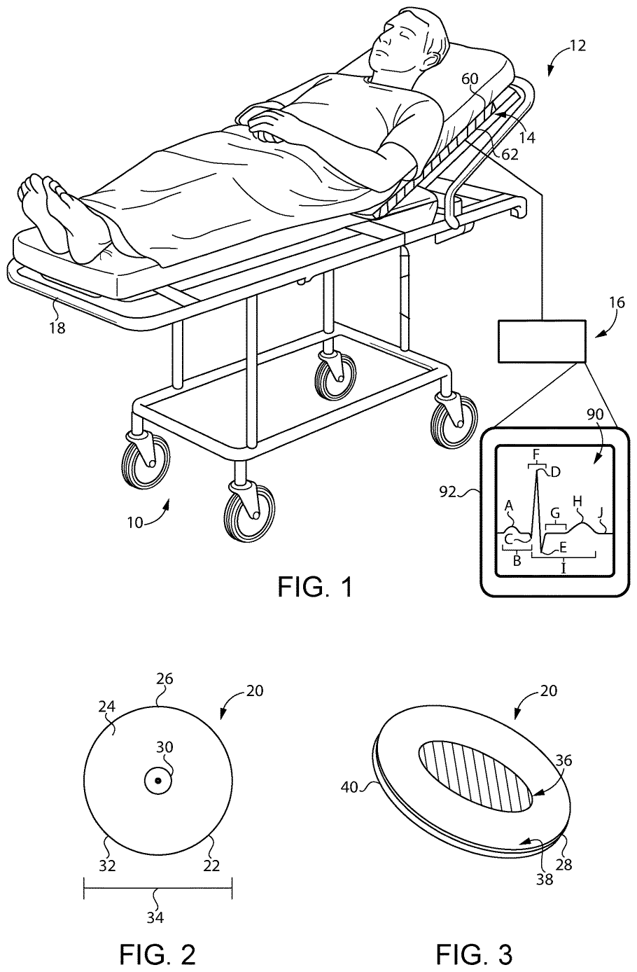

Referring to FIG. 1, there is shown a bioelectrical signal acquisition system 10, according to one embodiment. Bioelectrical signal acquisition system 10 may include an electrocardiogram system 12 (hereafter “ECG system 12”). ECG system 12 may include a cassette 14 connected to a base display unit 16. As illustrated, a human subject, hereafter referred to as a “patient”, is resting in close proximity to cassette 14. ECG system 12 may be used for collecting, recording, and processing bioelectrical signals, such as electrocardiographic signals, and may be operable in a number of clinical and non-clinical applications, including for instance, diagnostic evaluation, therapeutic monitoring, physiological data acquisition, and still other instances. ECG system 12 may be operable independent of the clinical condition, physiological status, or state of consciousness for any given patient. Put differently, the patient could be symptomatic or asymptomatic, ambulatory or non-ambulatory, and could be in a wakeful, drowsy, or asleep state at the time of ECG signal acquisition.

A support structure 18 may be provided for patient positioning at the time of ECG signal acquisition. As shown, support structure 18 may include a mobile stretcher or gurney, but is not limited to such, and alternative structures are contemplated. For example, support structure 18 could include an adjustable hospital bed, a procedural examination table, a wheelchair, a surgical table, or still something else, provided certain components remain in close proximity to the patient as further apparent from the forthcoming description.

Now also referring to FIGS. 2 and 3, ECG system 12 may also include one or more electrodes 20. It should be understood that the term “electrode” could include a plurality of electrodes used in a generally conventional manner to capture electrical signals from various anatomical locations, and could include a multi-lead configuration, such as 3-lead, 5-lead, or 12-lead arrangements. Electrode 20 will be discussed in the singular at least at times from hereinafter. Electrode 20 may be connected to cassette 14. The electrode-cassette connection may be achieved via wireless transmission interfaces, such as induction or the use of electromagnetic fields.

As will be further apparent from the forthcoming discussion, ECG system 12 may be equipped for generating, acquiring, processing, and evaluating a signal that is influenced by the interaction between an electromagnetic field and the bioelectrical characteristics of the patient's tissue in contact with or adjacent to, electrode 20. The resulting signal could include features that correlate with physiological activity and can be extracted through various signal processing methods for various purposes.

Electrode 20 may include an electrode body 22 having a first electrode side 24, and a second electrode side 26 having a contact pad 28 thereon. Contact pad 28 may be structured to establish an electrical connection between a patient and electrode 20 in a generally conventional manner. Electrode 20 may include an electrically conductive components, at least in part, and may be fabricated of a biocompatible material. Electrode 20 may also include a button 30 located on first electrode side 24. Button 30 may be an attachment location, which could allow for the connection of wired leads in instances where a direct wired connection is desirable.

As shown in FIG. 2, electrode body 22 may be planar with an electrode peripheral edge 32 forming a circular shape, however it is limited to such. Electrode 20 may also define a diameter dimension 34 linearly extending across the circular electrode body 22. In one practical example, diameter dimension 34 may be approximately 1 inch, but isn't limited to such and alternative sizes are contemplated. It should be understood that while the electrode of the present description includes a circular shape, other shapes are contemplated, such as oblong shapes, squares, or other shapes. As further apparent from the forthcoming discussion, electrode 20 may not constantly be hardwired. For instance, in some embodiments, electrode 20 may instead receive induced energy through field interactions. In other embodiments, electrode 20 may include a battery, wirelessly operable via charging, or some combination thereof.

Electrode 20 may also include an electrode disc 36 positioned in or coupled to electrode 20. Electrode disc 36 may form a circular shape and could, for example, provide a reference point for signal processing functions or for wireless communication. In one practical example, electrode disc 36 may receive and transmit biopotential signals from the surface of the skin and may be fabricated from a variety of conductive materials. Electrode 20 may also include a substrate 38 as a carrier for electrode disc 36 within electrode 20. It should be appreciated that electrode 20 could alternatively be strategically positioned to cooperate with support structure 18. For instance, a wheelchair as a support structure could include electrode interface panels. In a practical implementation, ECG system 12 may also include a conductive electrode gel substrate 40 to improve the quality or reliability of signal transmission or acquisition.

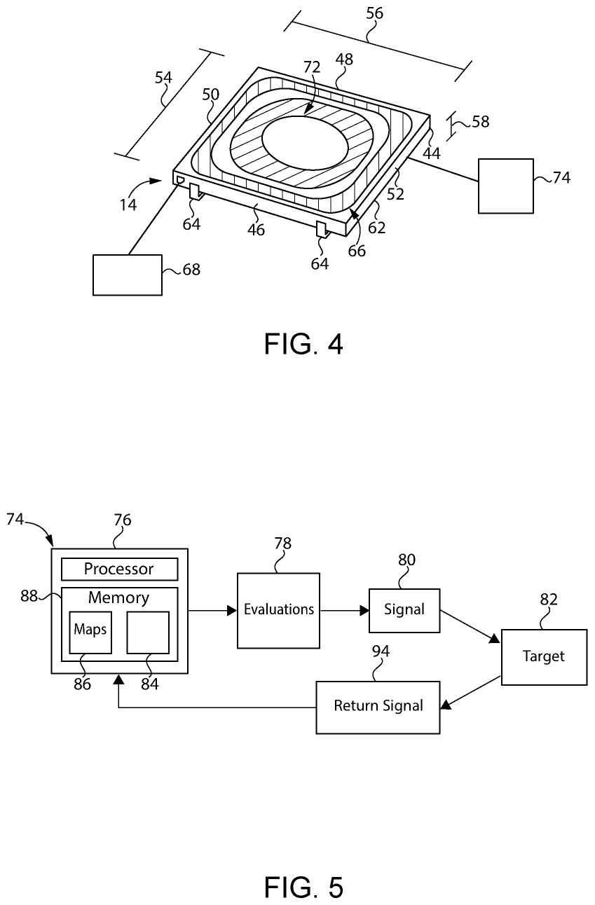

Now also referring to FIG. 4, there are shown additional features of cassette 14. Cassette 14 may include a modular cassette body 44 which could be fabricated from a non-ferrous material or other suitable materials with desirable electrical insulation properties, such as polymers. Cassette body 44 may have a proximal edge 46, a distal edge 48, a first lateral edge 50, and a second lateral edge 52. Cassette body 44 may have a rectangular configuration defining three orthogonal linear dimensions, a length dimension 54, a width dimension 56, and a height dimension 58. Length dimension 54 extends between proximal edge 46 and distal edge 48, width dimension 56 extends between first lateral edge 50 and second lateral edge 52, and height dimension 58 extends between an upper surface 60 and lower surface 62.

In one example, length dimension 54 may be approximately 18 to 20 inches, width dimension 56 may be approximately 20 to 22 inches, and height dimension 58 may be approximately 1 to 1½ inches, thereby forming a rectangular footprint. Cassette 44 may also include a clamping mechanism 64. It should be understood that the term “approximately” means within a tolerance or margin of variation, including a deviation from the stated value, as would be understood by one of ordinary skill in the art. As shown in FIG. 1, cassette 14 is shown beneath the patient, but alternative placement can be accomplished using one or more clamping mechanisms 64, such as upon a side rail of the gurney for example.

Cassette 44 may also include at least one energizing coil 66. Energizing coil 66 may be coupled to, or in the general proximity of cassette 44. In a practical example, energizing coil 66 may be located resident within cassette body 44. It should be appreciated that energizing coil 66 may be one of a plurality of coils resident in cassette 44. In a practical implementation, energizing coil 44 could be tapered and/or strategically positioned to produce more direct electromagnetic fields.

ECG system 12 may also include base display unit 16 connected to a power source 68. Base display unit 16 may be connected to cassette 14 using a cable 70. In a practical implementation, cable 70 may be shielded from electromagnetic fields utilized by ECG system 12 using, for instance, a thin film of aluminum. Power source 68 may be electrically coupled to energizing coil 66 via cassette 14. Power source 68 may include, for example, a standard alternating current (AC) outlet structured to provide alternating current to energizing coil 66. Alternatively, a direct current (DC) outlet could be utilized. The current may flow through energizing coil 66 to produce an electromagnetic field, hereafter “energizing electromagnetic field”. Energizing electromagnetic field may radiate outward from energizing coil 66 and can extend beyond the spatial region surrounding cassette 14, thereby forming an inductive region capable of transferring energy wirelessly, hereafter interchangeably termed “the region of influence”.

As suggested above, electrode 20 may be placed in physical contact with the surface of a patient's skin and can be situated in the region of influence of the energizing electromagnetic field. Electrodes 20 may be, for example, placed on one or a plurality of places of a patient's torso. Based on positioning in the region of influence the energizing electromagnetic field generates changes to electrical properties of electrode 20, including producing eddy currents according to well-known principles.

Cassette 14 may also include a sensing coil 72, such as an electromagnetic sensor or field-responsive transducer, structured to establish a sensing field. Sensing coil 72 may be located in proximity to the energizing field. Sensing coil 72 could additionally be located within cassette 14, and electronically coupled to power source 70 to be energized. It should be understood that the sensing field may be not an active field, but rather refers to the electromagnetic environment sensing coil 72 can detect. Sensing coil 72 may monitor, for example, perturbations or modulations in the eddy currents present in electrode 20. In this manner, each electrode 20 may act as a designated receiving magnet.

When a patient generates a bioelectric potential, such as a cardiac or neural potential, the resulting electrical activity may modulate the electrical potential at the contact interface of electrode 20. Another way to understand this principle is that the biopotential may influence the magnitude and/or disrupt the eddy currents, thereby causing detectable variations in the electrical activity of the electrode 20. Sensing coil 72 may be designed to detect perturbations in the eddy currents. For example, the variations could induce measurable voltage changes in sensing coil 72.

In a practical implementation, a clinician could, for instance, place at least one electrode 20 in electrical contact with the body of a patient. Upon activation, energizing coil 66 may be energized by a current, thereby generating an energizing field. The resulting energizing field may induce an electric current in electrode 20, which can be monitored to detect changes in electrical activity using sensing coil 72.

In some embodiments, to monitor multiple regions, a plurality of electrodes 20 may be placed across different locations of the patient. Each electrode 20 may be sequentially or simultaneously exposed to the energizing electromagnetic field. The sensing field may distinguish signals from each electrode 20 by using various strategies such as spatially resolved sensors or unique signal characteristics such as frequency tags.

In a practical example, the perturbations in the eddy currents can be interpreted as indicative of electrocardiographic waveforms, and can be observed via the sensing field. For example, perturbations in the sensing field may correspond to eddy current perturbations caused by depolarization and repolarization phases of the heart. In another example, the perturbations may be used to detect electromyographic signals (EMG) generated by skeletal muscle activation, such as during a muscle contraction. In other examples, electroencephalographic (EEG) activity arising from brain activity, or electrogastrographic (EGG) signals originating from smooth muscle contractions in the gastrointestinal tract could be detected, and still more.

ECG system 12 may also include a controller 74 and a processor 76 to execute operational instructions. Also referring now to FIG. 5, which illustrates how controller 74 may perform evaluations 78, and produce signals 80 or commands for communication with a target 82 as further discussed herein. To this end, ECG system 12 may include a communication module 84 for wireless control via Wi-Fi, Bluetooth, or Ethernet, for example, and one or more maps 86 structured to relate inputs to output signal or the like.

Controller 74 may execute software or firmware for task execution, data collection, control signal generation, and system monitoring, and still other tasks. In a practical implementation, controller 74 can be programmed to initiate diagnostic routines, manage data, and control each field produced. Controller 74 could, for example, be operably coupled to sensing coil 72 to extract signal components indicative of cardiac bioelectric activity from the received induced electromagnetic field. ECG system 12 may also include a memory 88 for various purposes, such as storage of ECG signal data, system parameters, diagnostic flags, and still other information.

Referring again now to FIG. 1, there is shown an exemplary ECG wave pattern 90. Typically, ECG wave patterns 92 include a P wave (A), a PR interval (B), a Q wave (C), an R wave (D), an S wave (E), a QRS complex (F), an ST segment (G), a T wave (H), a QT interval (I), and a U wave (J). P waves (A) usually represent atrial depolarization, QRS complex (F) represents ventricular depolarization, and T wave (H) represents ventricular repolarization, in a generally convention manner.

ECG system 12 could be used as a surrogate for real-time monitoring of electrophysiological cardiac parameters, including but not limited to heart rate, rhythm, regularity, and signal morphology, and still more. In a practical implementation, the observed electric properties of electrode 20 could be processed and subsequently displayed as a time dependent waveform corresponding to a conventional ECG signal on a monitor 92. The waveform could also include an isoelectric line (or baseline). Each waveform component could be used for various purposes, such as clinical assessments for detection of abnormalities. In some embodiments, data garnered from monitored bioelectrical activity or changes in patient bioelectrical activity may be stored, and used to establish a database, or feed into an existing database.

As mentioned above, ECG system 12 may gather data indicative of the ECG activity of the patient. The collected data can then be compared to an expected reference ECG data set, which may be derived from a normative database or prior-patient specific information, for instance. In the course of the comparison, deviations between the patient's ECG data and the expected ECG data may be identified. The deviations can be analyzed to determine potential abnormalities, which may indicate certain conditions such as arrhythmias, ischemia, or other cardiovascular conditions. The extent of these abnormalities could be quantified based on a magnitude and or pattern of the deviations. The comparative assessment of the patient's cardiac condition may reveal any significant deviations from expected normal cardiac function.

In practical implementation, cassette 14 could be configured to transmit a wireless signal encoding data related to sensed electrical activity. For instance, the wireless signal could encode a physiological or environmental datum. The signal could be emitted by cassette 14 and can be received by a receiving device, such as controller 74. In other embodiments, the receiving device could be a mobile computing device or a smartphone, which could be equipped with a software application capable of receiving, interpreting, decoding, anonymizing and/or storing received data. The software application could include functions to associate the transmitted data with specific patient profile, for instance.

Referring again now to FIG. 5, ECG system 12 may include a server in communication with controller 74. For instance, target 82 may be a server. The server may be a local server, or a remote server, such as on a network-accessible platform or other computing resource. Controller 74 may be configured to generate signals 80 corresponding to the detected activity, which may be sent to a hospital network including information for a plurality of patients. Signals 80 may represent raw measurements, conditioned data, extracted features, or other encoded information. In some embodiments, controller 74 can condition the signal prior to transmission, which may include reducing background noise, filtering static interference, compensating for artifacts, or otherwise enhancing signal quality to improve evaluation. Controller 74 may transmit signals 80 to targets 82 and may also be configured to receive a return signals 94.

The server may include an artificial intelligence engine configured to evaluate signals 80 and identify patterns, trends, or deviations associated with a patient. The artificial intelligence engine may include one or more machine-learned models, which may be trained to recognize recurring results in data, determine abnormalities, or account for variability amongst demographics of populations. The patterns identified by the artificial intelligence engine may correspond to temporal sequences, waveform characteristics, rate changes, or still some other change over time. In some embodiments, pattern recognition may be refined by incorporating historical data, prior evaluations, or feedback, for optimized subsequent analyses.

In a practical example, based on the identified patterns, ECG system 12 may infer a probable user condition and generate one or more outputs. Such outputs may include reports, summaries, classifications, or other forms of interpreted information. The reports may provide information about a patient, identify detected irregularities, indicate changes relative to baseline behavior, or provide contextual explanations or observed activity.

As indicated above, controller 74 may also receive return signals 94 from targets 82, such as a server. Such return signals 94 could include information derived from or based on evaluations 78. For example, return signal 94 say include a condition estimate, a prompt, a recommendation, a report, a command, an alert, a notification, or still some other command or signal. Return signals 94 may be configured to cause controller 74 or other components of ECG system 12 to output information. For instance, in a practical example, a communication may be provided to the patient, a physician, or device based on signals 80 or return signals 94 to trigger an escalation pathway. The term “escalation pathway,” as used in the present description denotes instances in clinical or non-clinical settings where the data received via signals 80 or return signal 94 requires attention. This could include, for example, drastic changes in a condition, changes consistent with predetermined circumstances, or other instances. By way of example, an alert or notification may indicate that the detected physiological activity is consistent with deviation from expected patterns, or that further interaction is warranted.

### INDUSTRIAL APPLICABILITY

It will be recalled that operating a wireless ECG system 12 may include generating an energizing electromagnetic field to induce an electric current in electrode 20 attached to a patient. Operating ECG system 12 also includes monitoring a sensing electromagnetic field to detect a change in an electrical property of electrode 20 attached to the patient. Operating ECG system also includes producing the signal indicative of electrocardiographic activity of the patient based on the change detected in the sensing electromagnetic field. ECG system 12 may also include a monitor 92, for real-time display.

The present description is for illustrative purposes only, and should not be construed to narrow the breadth of the present disclosure in any way. Thus, those skilled in the art will appreciate that various modifications might be made to the presently disclosed embodiments without departing from the full and fair scope and spirit of the present disclosure. Other aspects, features and advantages will be apparent upon an examination of the attached drawings and appended claims. As used herein, the articles “a” and “an” are intended to include one or more items, and may be used interchangeably with “one or more.” Where only one item is intended, the term “one” or similar language is used. Also, as used herein, the terms “has,” “have,” “having,” or the like are intended to be open-ended terms. Further, the phrase “based on” is intended to mean “based, at least in part, on” unless explicitly stated otherwise.

1. A method for operating a wireless electrocardiogram comprising:
generating an energizing electromagnetic field to induce an electric current in an electrode attached to a patient;
wirelessly detecting a change in an electrical property of the electrode attached to the patient; and
producing a signal indicative of electrocardiographic activity of the patient based on the detected change.

2. The method of claim 1 further including gathering data indicative of the electrocardiographic activity of the patient.

3. The method of claim 2 further including comparing the data indicative of the electrocardiographic activity of the patient to expected electrocardiographic data.

4. The method of claim 3 further including determining an abnormality based on the comparing the data.

5. The method of claim 4 further including generating an alert based on the determining an abnormality based on the comparing the data.

6. The method of claim 1 further including activating an energizing coil so as to generate the energizing electromagnetic field.

7. The method of claim 6 wherein the induced electric current includes an eddy current.

8. The method of claim 7 wherein the change in the electrical property includes a perturbation in the eddy current.

9. The method of claim 8 wherein the perturbation is based on bioelectric activity of the patient.

10. The method of claim 1 further including displaying on a monitor the electrocardiographic activity of the patient.

11. A method for detecting bioelectrical activity comprising:
placing at least one electrode in direct electrical contact with the body of a patient;
energizing an electromagnetic coil to wirelessly induce electric current in the at least one electrode;
monitoring a sensing electromagnetic field; and
wirelessly detecting a change in bioelectrical activity of the body of the patient based on the monitoring the sensing electromagnetic field.

12. The method of claim 11 wherein the change in the bioelectrical activity of the patient is associated with depolarization of an excitable tissue in the patient.

13. The method of claim 11 wherein the induced electric current includes an eddy current.

14. The method of claim 11 further including producing a signal corresponding to the biopotential of the patient on a display unit.

15. The method of claim 11 further comprising wirelessly monitoring patient bioelectrical activity, and storing one or more of the monitored patient bioelectrical activity or the change in bioelectrical activity.

16. The method of claim 15 further comprising establishing a database based on at least one of the monitored patient bioelectrical activity or the change in bioelectrical activity.

17. An electrocardiogram system comprising:
an energizing coil configured to produce an electromagnetic field;
one or more contact pads structured for attaching to a patient; and
a sensing coil structured to wirelessly monitor electrical activity of the contact pads induced by an electromagnetic field produced by the energizing coil.

18. The electrocardiogram system of claim 16 wherein the energizing coil and the sensing coil are housed in a cassette.

19. The electrocardiogram system of claim 16 further comprising a controller operably coupled to the sensing coil and configured to transmit signals to a remote server based on the wirelessly monitored electrical activity.

20. The electrocardiogram system of claim 16 wherein the monitored electrical activity is indicative of P waves, QRS complexes, and T waves.
